(19) United States
(12) Patent Application Publication
(10) Pub. No.: US 2025/0273340 A1
(43) Pub. Date: Aug. 28, 2025

(54) SYSTEM FOR PROVIDING IMMUNE STATUS PREDICTION, AND METHOD AND PROGRAM FOR PREDICTING IMMUNE STATUS DATA

(71) Applicant: Edgewater Corp., Setagaya-ku, Tokyo (JP)

(72) Inventor: Masahiko FUKUZAWA, Setagaya-ku, Tokyo (JP)

(21) Appl. No.: 18/850,822

(22) PCT Filed: May 17, 2022
(86) PCT No.: PCT/JP2022/020513

§ 371 Date: May 16, 2025

(51) Int. Cl. G16H 50/30; G16H 15/00

(52) U.S. Cl. CPC G16H 50/30; G16H 15/00

(57) ABSTRACT

[Problem] To make it possible to predict an immune state in the future by assessing a factor of an immune state as well as an existing health examination and a lifestyle habit. [Solution] An immune state prediction provision system 1 is configured such that at least one item of state data associated with an immune state which is selected from a sleep state, a motion state, a lifestyle habit state, a life state, a work state and a meal state is acquired, then a learning model that generates immune state prediction data is produced from the acquired state data, and then the immune state is predicted on the basis of the learning model from newly acquired state data. Subsequently, the difference between the immune state prediction data and execution data such as the results of a health examination and the results of an immunity test is analyzed to produce analysis result data, and the analysis result data is output. In addition, at least one item of data selected from the state data, the immune state prediction data, the execution data and the analysis result data are provided for a user or a third person.

### TECHNICAL FIELD

The present invention relates to a system for providing immune status prediction, and a method and a program for predicting immune status data.

### BACKGROUND

In recent years in Japan, the increase in life expectancy and the impact of an aging population with fewer children have led to concerns about the growing burden of social security costs on the real economy. Thus, not only the promotion of healthy life expectancy after retirement, but also the focus on health management for active workers by companies is attracting significant attention. The movement to contribute to the improvement of health status and the prevention of diseases by making better use of the results of regular health checkups, including employer-provided examinations can be observed.

In health management, health checkups are used to understand the health status. However, there is a problem of insufficient information about lifestyle habits causing health status and about the immune status related to the process. Therefore, there is a demand for technology that can collect more accurate status data by associating various information including lifestyle habits.

For example, a system has been disclosed that conducts interviews about lifestyle habits, compares the lifestyle habits with the results of health checkups, and predicts the past and current lifestyle habits and the future status (Patent Document 1).

### DOCUMENT IN THE EXISTING ART

### Patent Document

Patent Document 1: JP 2020-101843 A

### SUMMARY

### The Technical Problem Solved by the Disclosure

The technology described in Patent Document 1 can acquire rough data related to lifestyle habits which are some of the causes of health status. However, the technology is not able to understand the immune status which is important for health management because the acquired data lacks the cause of immune status.

Generally, it is known that immunity is important in the incidence and recovery of infectious diseases and cancer (malignant neoplasms). Due to the recent global epidemic of infectious diseases, the focus on immunity has increased, and the demand for functional foods labelled as an immune functional food is also increasing. However, traditional health checkups do not include immunity tests. Improving the immune status involves understanding sleep status, exercise status, lifestyle habits, living status, working status, and dietary status. However, no system to comprehensively collect and analyze these data exists.

An objective of the present invention is to provide a system for providing immune status prediction that predicts future immune status from user status data, which is capable of the prediction of future immune status by understanding status data that includes not only traditional health checkups and lifestyle habits but also sleep status, exercise status, lifestyle habit status, living status, working status, and dietary status which are factors of the immune status.

### Solution for Solving the Technical Problem

The present invention provides the following solution.

A first aspect of the present invention provides a system for providing immune status prediction including:
an acquisition unit that acquires at least one of the status data of sleep status, exercise status, lifestyle habit status, living status, working status, and dietary status related to an immune status;
a learning model creation unit that creates a learning model to generate immune status prediction data from the acquired status data;
a prediction unit that predicts immune status prediction data based on the learning model from newly acquired status data;
an analysis unit that generates analysis result data by analyzing the difference between the immune status prediction data and implementation data including a health checkup result and an immune test result;
a first output unit that outputs the predicted immune status prediction data;
a second output unit that outputs the generated analysis result data; and
a provision unit that provides at least one of the status data, the immune status prediction data, the implementation data, and the analysis result data to a user or a third party.

The first aspect of the present invention can create a learning model that predicts future immune status based on the acquired status data and user's future immune status from the newly acquired user status data based on the learning model. The first aspect of the present invention can also analyze the cause of the difference between the time of acquiring user's status data and the time of acquiring the implementation data by comparing the predicted immune status prediction data and the implementation data such as a user's health checkup and an immune test.

The first aspect of the present invention can also provide at least one of the status data, the immune status prediction data, the implementation data, and the analysis result data to a user or a third-party company.

A second aspect of the present invention according to the first aspect of the present invention includes a learning model creation unit that creates a learning model that creates immune status prediction data from the implementation data and the previous status data.

According to the second aspect of the present invention, the system for providing immune status prediction according to the first aspect of the present invention can improve the prediction accuracy of user's future immune status by creating a learning model from the status data and the implementation data.

In a third aspect of the present invention according to the second aspect of the present invention, the provision unit provides at least one of the user attribute data, the status data, the immune status prediction data, the implementation data, and the analysis result data to a third-party company according to a user attribute preset by the third-party company.

According to the third aspect of the present invention, the system for providing immune status prediction according to the second aspect of the present invention can provide data according to the demands of a third party company.

A fourth aspect of the present invention provides the system for providing immune status prediction according to the second aspect of the present invention further includes a message generation unit that generates a message for joint development to the third-party company in response to a request received from the third-party company;
a message provision unit that provides the message to the third-party company; and
a message reception unit that receives the message from the third-party.

According to the fourth aspect of the present invention, the system for providing immune status prediction according to the second aspect of the present invention can further improves the prediction accuracy of the immune status by promoting joint development.

A fifth aspect of the present invention provides the system for providing immune status prediction according to the first or the second aspect of the present invention further includes a standardized index creation unit that creates a standardized index from the user attribute data, the status data, the immune status prediction data, the implementation data, and the analysis result data; and
a standardized index provision unit that provides the standardized index to the third-party company.

According to the fifth aspect of the present invention, the system for providing immune status prediction according to the first or the second aspect of the present invention provides a specific standardized index to improve user's immune status.

The first aspect of the present invention is a category of system, but also achieved in the categories of method and program, and achieves the structure, action, and effect in each category.

### Technical Effect

The present invention provides a system for providing immune status prediction, method, and program that can understand user's immune status including the cause and perform more effective health management by predicting the future immune status.

### BRIEF DESCRIPTION OF THE DRAWINGS

FIG. 1 shows an overview of the system for providing immune status prediction 1.

FIG. 2 is a configuration diagram of the system for providing immune status prediction 1.

FIG. 3 is a flowchart showing the procedure of the creation process performed by the computer 2 of the system for providing immune status prediction 1.

FIG. 4 is an example display screen of sleep status data of the status data 102 acquired by the computer 2, which is displayed on the user terminal 3.

FIG. 5 is an example display screen of immune status prediction of the immune status prediction data 104 created by the computer 2, which is displayed on the user terminal 3.

FIG. 6 is a flowchart showing the procedure of the analysis result data generation process performed by the computer 2 of the system for providing immune status prediction 1.

FIG. 7 is an example display screen of the immune test result of the implementation data 103 acquired by the computer 2, which is displayed on the user terminal 3.

FIG. 8 is an example analysis display screen of the analysis result data 105 created by the computer 2, which is displayed on the user terminal 3.

FIG. 9 is a flowchart showing the procedure of the learning model improvement process performed by the computer 2 of the system for providing immune status prediction 1.

FIG. 10 is a flowchart showing the procedure of the selective data provision process performed by the computer 2 of the system for providing immune status prediction 1.

FIG. 11 is a configuration diagram of the joint development promotion process performed by the computer 2 of the system for providing immune status prediction 1.

FIG. 12 is a flowchart showing the procedure of the joint development promotion process performed by the computer 2 of the system for providing immune status prediction 1.

FIG. 13 is a configuration diagram of the standardized index creation process performed by the computer 2 of the system for providing immune status prediction 1.

FIG. 14 is a flowchart showing the procedure of the standardized index creation process performed by the computer 2 of the system for providing immune status prediction 1.

### DETAILED DESCRIPTION

The present invention will be more specifically described below with reference to the preferable embodiments. However, these are illustrative only, and the present disclosure is not limited thereto.

### Overview of the System for Providing Immune Status Prediction 1

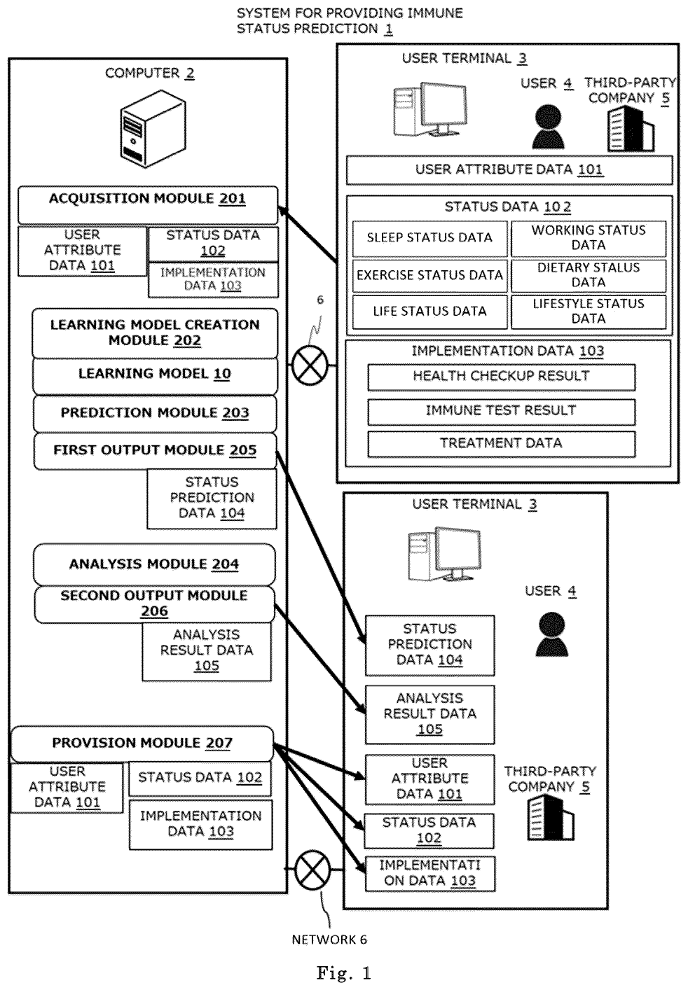

FIG. 1 is a diagram for explaining the overview of the system for providing immune status prediction 1. The overview of the system for providing immune status prediction 1 is explained below with reference to FIG. 1.

As shown in FIG. 1, the system for providing immune status prediction 1 is a computer system used for predicting immune status, including a computer 2 and a user terminal 3.

The computer 2 of the system for providing immune status prediction 1 may be a server such as a cloud server or may be a general personal computer or laptop.

The user terminal 3 of the system for providing immune status prediction 1 is a terminal for transmitting and receiving status data and implementation data to and from the computer 2. The user terminal 3 may be a personal computer or laptop, a mobile terminal such as a smartphone or tablet, or a wearable terminal such as a head-mounted display like smart glasses or a smartwatch.

The computer 2 of the system for providing immune status prediction 1 may be achieved by one or more computers physically or by a virtual device like a cloud computer.

The number of user terminals 3, users 4, and third-party companies 5 may be more than one.

The computer 2 of the system for providing immune status prediction 1 may be data-communicatively connected to the user terminal 3 and a network 6 such as a public line network and may transmit and receive necessary data and information.

The computer 2 of the system for providing immune status prediction 1 enables the prediction of future immune status by performing the respective processes of an acquisition module 201 that acquires at least user attribute data 101, status data 102, and implementation data 103 from the user terminal 3;
a learning model creation module 202 that creates a learning model 10 that generates data to predict future immune status from the status data 102 or from the status data 102 and the implementation data 103;
a prediction module 203 that predicts immune status prediction data 104 based on the learning model 10 from the implementation data 103;
a first output module 205 that outputs the predicted immune status prediction data 104;
an analysis module 204 that analyzes the difference between the output immune status prediction data 104 and the implementation data 103 to generate analysis result data 105;
a second output module 206 that outputs the generated analysis result data 105; and
a provision module 207 that provides at least one of the acquired user attribute data 101, status data 102, implementation data 103, output immune status prediction data 104, and analysis result data 105 to a user 4 or a third-party company 5 via the user terminal 3.

The user attribute data 101 refers to data that includes at least attribute data such as user's age, gender, height, weight, hobby, educational background, work history, or family composition.

The status data 102 includes at least sleep status data such as user's sleep duration over a certain period, sleep depth, or wake-up frequency during sleep; exercise status data such as the number of steps, exercise frequency, or exercise duration; lifestyle status data such as smoking frequency, drinking frequency, or the amount of alcohol consumed; life status data such as active hours or sleeping hours; working status data such as working hours or work content; and dietary status data such as meal content, snack frequency, calorie intake, or nutritional balance.

The implementation data 103 includes at least health checkup data of the health checkup that a user actually had at a certain timing (over a certain period), immune test data of the immune test that a user actually had at a certain timing (over a certain period), and treatment data indicating that a user actually received treatment at a certain timing (over a certain period).

The status data 102, the implementation data 103, the immune status prediction data 104, and the analysis result data 105 may be stored inside or outside the computer 2, each associating with user attribute data.

This is an overview of the system for providing immune status prediction 1

### System Configuration of the System for Providing Immune Status Prediction 1

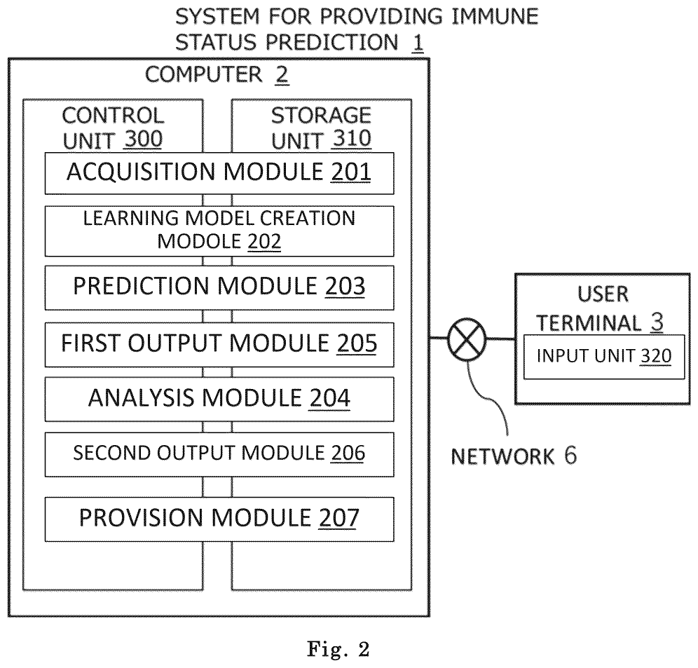

FIG. 2 is a diagram for explaining the system configuration of the system for providing immune status prediction 1. The system configuration of the system for providing immune status prediction 1 is explained below with reference to FIG. 2.

The computer 2 of the system for providing immune status prediction 1 is provided with a control unit 300 including a CPU (Central Processing Unit), GPU (Graphics Processing Unit), RAM (Random Access Memory), and ROM (Read Only Memory). The control unit 300 achieves the acquisition module 201, learning model creation module 202, prediction module 203, analysis module 204, first output module 205, second output module 206, and provision module 207 in cooperation with the storage unit 310.

The computer 2 is provided with data storage such as a hard disk, a semiconductor memory, a recording medium, or a memory card as a storage unit 310. The data may be a cloud service, a database, etc.

The user terminal 3 is provided with an input unit 320 with the necessary function to operate the computer 2. The user terminal 3 can be provided with, for example, a liquid crystal display that achieves touch panel functionality, a keyboard, a mouse, a pen tablet, hardware buttons on the device, and/or a microphone for voice recognition, for input function. The present invention is not particularly limited in its functions by any input method. This is the system configuration of the system for providing immune status prediction 1.

### Immune Status Prediction Data Creation Process

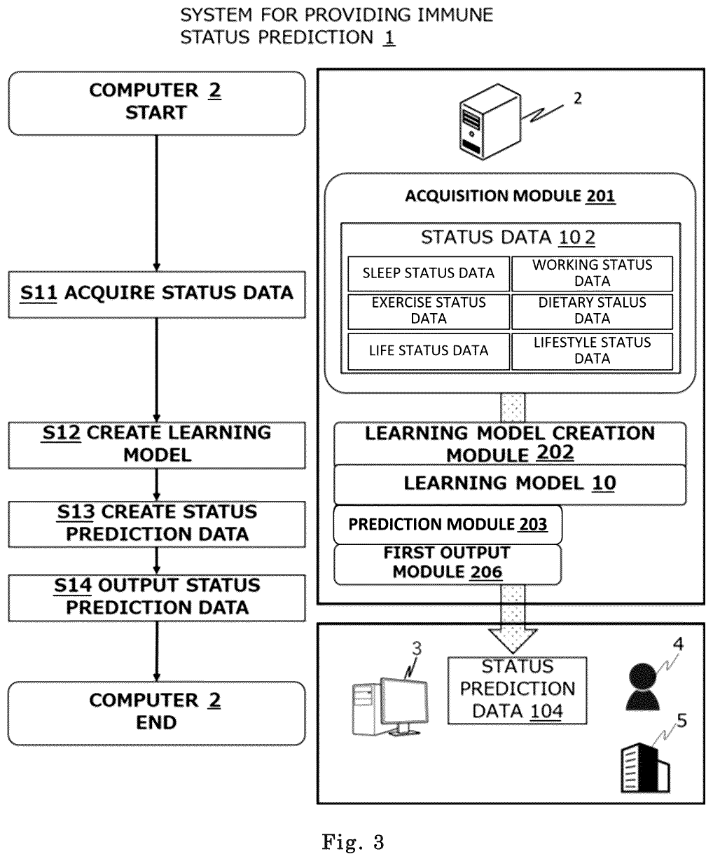

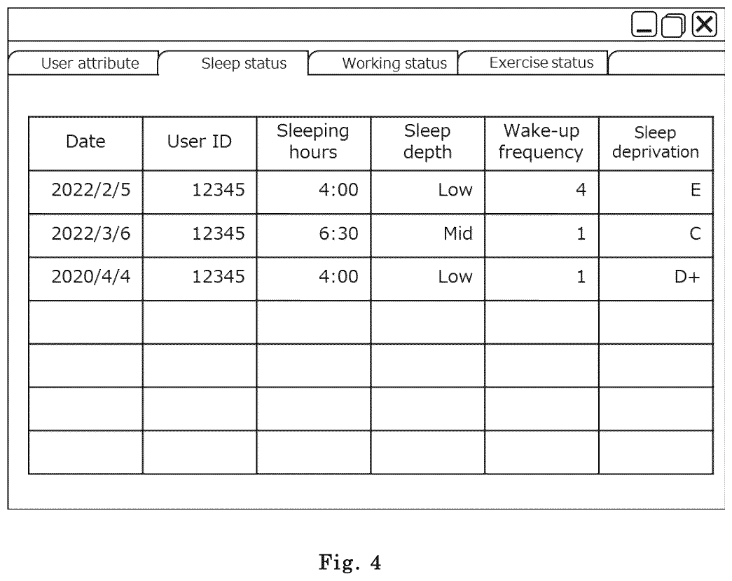

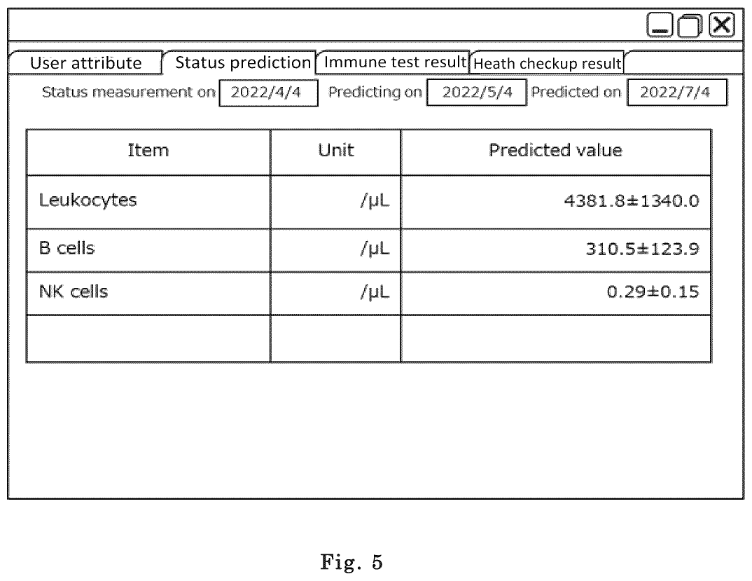

FIG. 3 is a diagram for explaining the immune status prediction data creation process performed by the computer 2 of the system for providing immune status prediction 1. FIG. 4 is an example display screen of sleep status data of the status data 102 acquired by the computer 2, which is displayed on the user terminal 3. FIG. 5 is an example display screen of immune status prediction of the immune status prediction data 104 created by the computer 2, which is displayed on the user terminal 3. The immune status prediction data creation process performed by the computer 2 of the system for providing immune status prediction 1 is explained below with reference to FIGS. 3 to 5.

The acquisition module 201 of the computer 2 acquires at least the status data 102 (Step S11).

The status data 102 refers to data that includes at least sleep status data, exercise status data, lifestyle habit status data, living status data, working status data, and dietary status data of a user 4 for a certain period of time as mentioned above. The format of the status data 102 includes all formats such as images, tables, numbers, and text, but is not limited thereto. The method of acquiring the status data 102 is not limited to the user terminal 3. The status data 102 may also be acquired from other terminal devices through a public line, etc. The timing of acquiring the status data 102 has no limitations. For example, the acquisition module 201 of the computer 2 may acquire only user's sleep status data for a certain period from the user terminal 3.

The acquired status data 102 may be stored inside or outside the computer 2.

The learning model creation module 202 of the computer 2 creates a learning model 10 from the status data 102 (Step S12). The learning model 10 created at this time may be created by annotating cases related to health status including diseases and immune status data as annotation data. The annotation data is teacher data used to train a machine learning model, which is annotated as information related to the status data 102 to combine data with each other by attaching a meaning to data and associating data with each other.

The creation of the learning model 10, for example, is carried out by annotating the sleep status data of the status data 102 with changes in the number of cells involved in immunity as annotation data for status prediction, based on scientific findings such as significant differences observed in the number of cells (e.g., B cells, some NK cells, leukocytes) involved in immunity that correspond to immune test results due to changes in sleep status.

The method of annotating data is not particularly limited and may be annotated by a manual method or by using a tagging automation tool such as an annotation tool.

The computer 2 creates immune status prediction data 104 which predicts future immune status based on the acquired user's status data 102 for a certain period based on the learning model 10 (Step S13).

Any period or any status data may be selected from the acquired status data 102 to predict the future immune status. The time of prediction may optionally be set. For example, as shown in FIG. 5, status prediction two months after the actual measurement of the sleep status data may be conducted by selecting the sleep status data from the status data 102.

The first output module 205 of the computer 2 outputs at least the predicted immune status prediction data 104 to the user terminal 3 (Step S14).

The predicted immune status prediction data 104 may be stored inside or outside the computer 2.

Accordingly, the system for providing immune status prediction 1 can mechanically perform status prediction for a vast number of patterns by using the learned data to predict future immune status from the acquired status data 102.

This is the immune status prediction data creation process executed by the system for providing immune status prediction 1.

### Analysis Result Data Generation Process

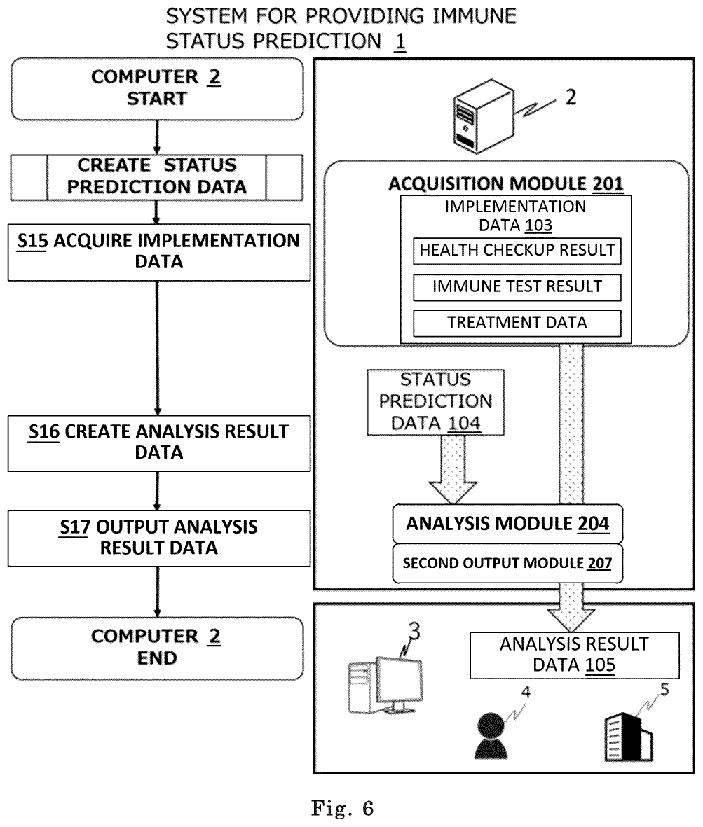

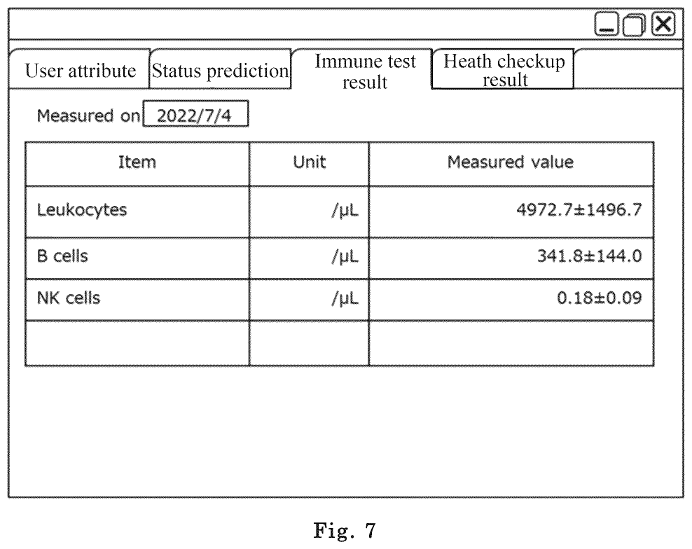

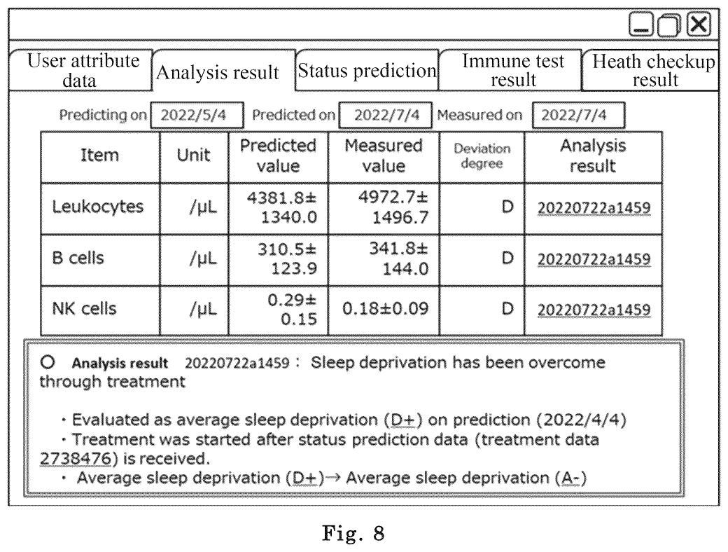

FIG. 6 is a diagram for explaining the analysis result data generation process performed by the computer 2 of the system for providing immune status prediction 1. FIG. 7 is an example display screen of the immune test result of the implementation data 103 acquired by the computer 2, which is displayed on the user terminal 3. FIG. 8 is an example analysis display screen of the analysis result data 105 created by the computer 2, which is displayed on the user terminal 3. The analysis result data generation process performed by the computer 2 of the system for providing immune status prediction 1 is explained below with reference to FIGS. 6 to 8.

Since the immune status prediction data creation process is the same as the above-mentioned immune status prediction data creation process, the explanation is omitted.

The acquisition module 201 of the computer 2 acquires at least the implementation data 103 from the user terminal 3 (Step S15).

The implementation data 103 is of the same user to which the status data 102 belongs acquired in the above-mentioned immune status prediction data creation process, which includes at least the health checkup result, immune test result, and treatment data of the user. For instance, FIG. 7 shows the actual measured number of the white blood cells, B cells, and NK cells in the immune test result of the user's implementation data 103. The format of the implemented data 103 includes all formats such as images, tables, numbers, and text, but is not limited thereto. The method of acquiring the implement data 103 has no limitations. The implement data 103 may also be acquired from other terminal devices through a public line, etc. The timing of acquiring the implement data 103 has no limitations.

The analysis module 204 of the computer 2 analyzes the difference between the acquired implementation data 103 and the created immune status prediction data 104 to generate analysis result data 105 (Step S16).

The immune status prediction data 104, which is created from the status data 102 of the same user to which the implementation data 103 belongs by the above-mentioned immune status prediction data creation process, indicates the immune status prediction data 104 created from the status data 102 acquired before the timing when the implementation data 103 is generated.

The analysis of the difference between the implementation data 103 and the immune status prediction data 104 involves analyzing the cause of discrepancy due to the time course between the prediction and the actual situation. The discrepancy arises at the point when the implementation data 103 is generated and the point when the status data 102 used to create the immune status prediction data 104 is generated.

For example, FIG. 8 shows the result of analyzing the difference between the actual measured number of the white blood cells, B cells, and NK cells in the immune test result of the user's implementation data 103 and the predicted values of the same items related to immunity in the immune status prediction data 104. The cause of the discrepancy is displayed as the result of the analysis, indicating that the discrepancy is caused due to the initiation of treatment upon receiving the presentation of the immune status prediction data 104.

The method of analyzing the cause is not particularly limited. For example, the cause can be analyzed using a rule-based or model-based method by machine learning, a manual input method, or a method using tagging automation tool such as an annotation tool.

The second output module 206 of the computer 2 outputs the generated analysis result data 105 to at least the user terminal 3 (Step S17).

The generated analysis result data 105 may be stored inside or outside the computer 2.

The system for providing immune status prediction 1 can clarify the changes from the time of status prediction to the time of implementation data measurement, including the cause, by analyzing the difference between the implementation data 103 and the immune status prediction data 104 and can contribute to more effective health management.

This is the analysis result data generation process.

### Learning Model Improvement Process

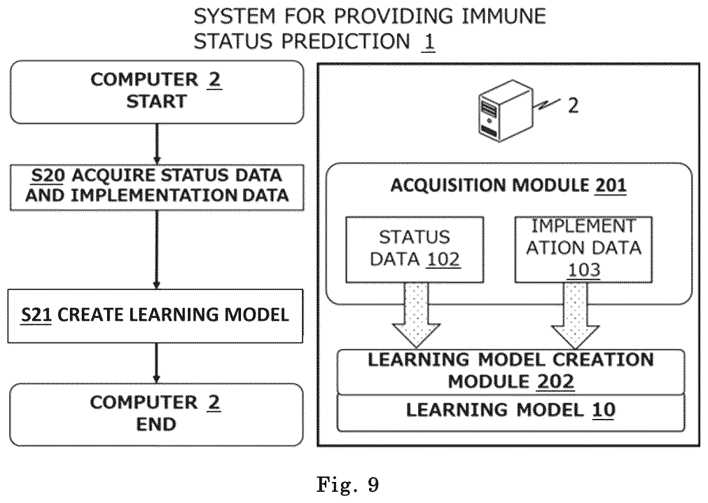

FIG. 9 is a diagram for explaining the learning model improvement process performed by the computer 2 of the system for providing immune status prediction 1. The learning model improvement process performed by the computer 2 of the system for providing immune status prediction 1 with reference to FIG. 9.

The learning model creation process is to improve the prediction accuracy of the learning model 10 which generates immune status prediction data for predicting future immune status, in addition to the method of creating the learning model 10 in the above-mentioned immune status prediction data creation process.

The acquisition module 201 of the computer 2 acquires at least the status data 102 and the implementation data 103 from the user terminal 3 (Step S20).

The implementation data 103 refers to the implementation data 103 of the same user as that of the acquired status data 102, which indicates the implementation data 103 created after the point when the status data 102 was created.

The implementation data 103 includes at least the health examination result, immune test result, and treatment data of the user. The format of the implemented data 103 includes all formats such as images, tables, numbers, and text, but is not limited thereto.

The method of acquiring the implement data 103 has no limitations. The implement data 103 may also be acquired from other terminal devices through a public line, etc. The timing of acquiring the implement data 103 has no limitations.

The learning model creation module 202 of the computer 2 creates a learning model 10 from the acquired status data 102 and implementation data 103 (Step S21).

The status data 102 acquired at this time is for machine learning. The implementation data 103 is teacher data for training the machine learning model, which is acquired as annotation data. The implementation data 103, as annotation data, trains the correlation for the prediction module 203 to generate the immune status prediction data 104 from the status data 102.

The status data 102 with annotation data added is machine-learned as the learning model 10.

Thus, the accuracy of the prediction based on the learning model 10 can be improved by training the learning model 10 with the status data 102 and the implementation data 103 of the same user. Accordingly, the present invention can mechanically perform status prediction for a vast number of patterns by using the learned data to predict future immune status from the acquired status data 102 to improve the accuracy of the cause analysis. This is the learning model improvement process.

### Selective Data Provision Process

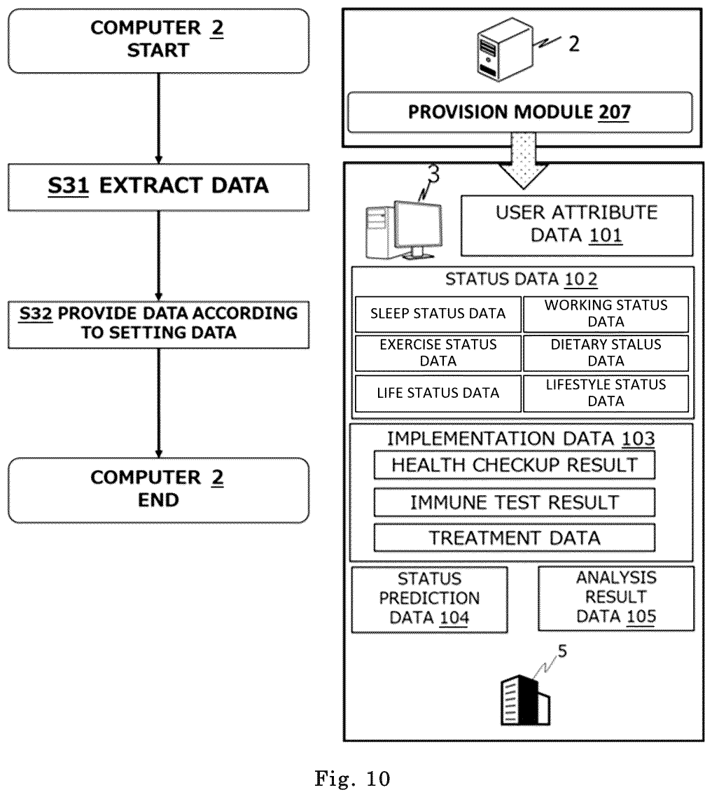

FIG. 10 is a diagram for explaining the selective data provision process performed by the computer 2 of the system for providing immune status prediction 1. The selective data provision process performed by the computer 2 of the system for providing immune status prediction 1 is explained below with reference to FIG. 6.

The provision module 207 of the computer 2 at least extracts the selective data from the user attribute data 101, status data 102, implementation data 103, immune status prediction data 104, and analysis result data 105 based on the user attributes preset by a third party company 5 (Step S31).

At least the setting data, user attribute data 101, status data 102, implementation data 103, immune status prediction data 104, and analysis result data 105 are assumed to have been acquired in advance in the memory unit 310 of the computer 2. The method of acquiring the setting data has no limitations. The setting data may also be acquired from other terminal devices through a public line, etc. The timing of acquiring the setting data has no limitations.

The provision module 207 of the computer 2 provides the extracted data to the third-party company 5 via the user terminal 3 (Step S32).

Accordingly, the system for providing immune status prediction 1 can, for example, accumulate trends in the information required by the third-party company 5 by providing the data to the third-party company 5 based on the setting data preset by the third-party company 5.

This is the selective data provision process.

### Joint Development Promotion Process

The joint development promotion process performed by the system for providing immune status prediction 1 is to enrich various data by conducting joint development with a third-party company and to improve the accuracy of the system for providing immune status prediction 1.

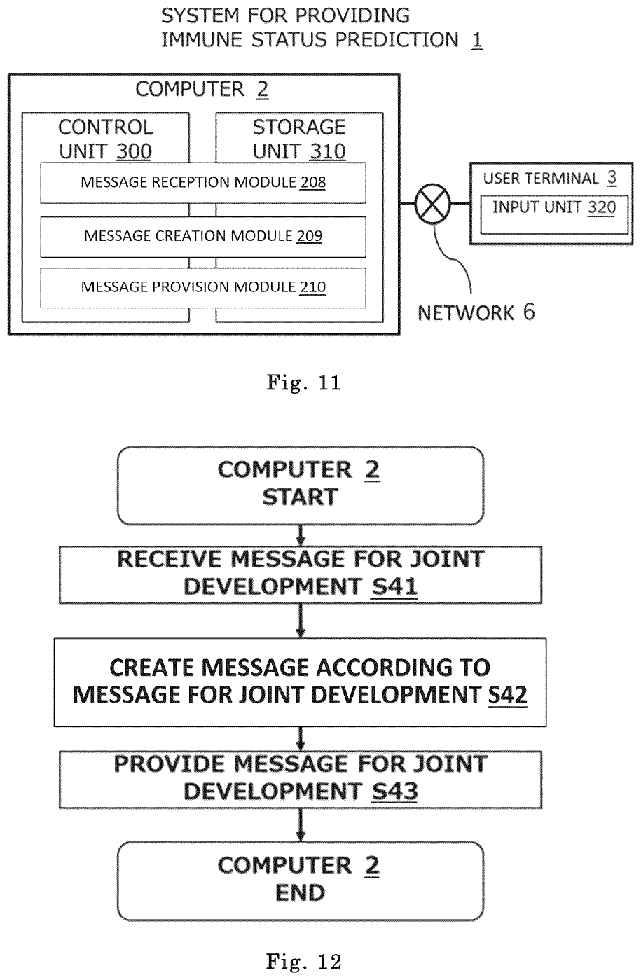

FIG. 11 is a configuration diagram of the joint development promotion process performed by the computer 2 of the system for providing immune status prediction 1. FIG. 12 is a flowchart showing the procedure of the joint development promotion process performed by the computer 2 of the system for providing immune status prediction 1.

The joint development promotion process performed by the computer 2 of the system for providing immune status prediction 1 is explained with reference to FIGS. 7 to 8.

The joint development promotion process performed by the computer 2 of the system for providing immune status prediction 1 is achieved by the computer 2, the user terminal 3, and the network 6 that connects the computer 2 with the user terminal 3.

The computer 2 that performs the joint development promotion process of the system for providing immune status prediction 1 is provided with a control unit 300 including a CPU (Central Processing Unit), GPU (Graphics Processing Unit), RAM (Random Access Memory), and ROM (Read Only Memory). The control unit 300, achieves the message reception module 208, the message creation module 209, and the message transmission module 210 in cooperation with the storage unit 310.

The user terminal 3 is provided with an input unit 320 with the necessary function to operate the computer 2. The user terminal 3 can be provided with, for example, a liquid crystal display that achieves touch panel functionality, a keyboard, a mouse, a pen tablet, hardware buttons on the device, and/or a microphone for voice recognition, for input function. The present invention is not particularly limited in its functions by any input method.

The computer 2 is provided with data storage such as a hard disk, a semiconductor memory, a recording medium, or a memory card as a storage unit 310. The data may be a cloud service, a database, etc.

The message reception module 208 of the computer 2 receives a message for joint development from the terminal of a company wishing joint development (Step S41).

The method of receiving a message for joint development has no limitations. The message may also be received from other terminal devices through a public line, etc. The timing of receiving the message has no limitations.

The message creation module 209 of the computer 2 creates a message according to the received message for joint development or a message inputted from the user terminal 3 according to a request (Step S42).

The method of inputting for the creation of a message for joint development is not particularly limited. The message may be manually input, or a preset standard message may be automatically input.

The message transmission module 210 of the computer 2 sends the created message to the terminal of the third-party company (Step S43).

The method of transmitting a message for joint development has no limitations. The message may also be transmitted to other terminal devices through a public line, etc. The timing of transmitting the message has no limitations. This is the joint development promotion process.

### Standardized Index Creation Process

The standardized index creation process performed by the computer 2 of the system for providing immune status prediction 1 is to create an index needed to evaluate or improve the user's immune status and provide the index to a third-party company.

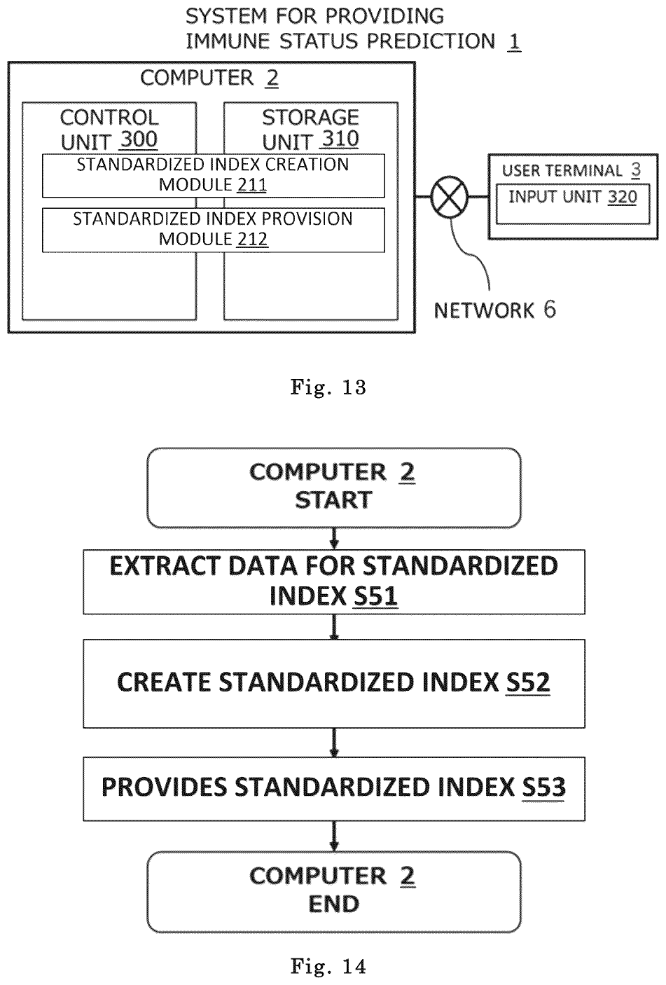

FIG. 13 is a configuration diagram of the standardized index creation process performed by the computer 2 of the system for providing immune status prediction 1. FIG. 14 is a flowchart showing the procedure of the standardized index creation process performed by the computer 2 of the system for providing immune status prediction 1.

The standardized index creation process performed by the computer 2 of the system for providing immune status prediction 1 is explained below with reference to FIGS. 13 to 14.

The standardized index creation process performed by the computer 2 of the system for providing immune status prediction 1 is achieved by the computer 2, the user terminal 3, and the network 6 that connects the computer 2 with the user terminal 3.

The computer 2 that performs the joint development promotion process of the system for providing immune status prediction 1 is provided with a control unit 300 including a CPU (Central Processing Unit), GPU (Graphics Processing Unit), RAM (Random Access Memory), and ROM (Read Only Memory). The control unit 300 achieves the standardized index creation module 211 and the standardized index provision module 212.

The user terminal 3 is provided with an input unit 320 with the necessary function to operate the computer 2. The user terminal 3 can be provided with, for example, a liquid crystal display that achieves touch panel functionality, a keyboard, a mouse, a pen tablet, hardware buttons on the device, and/or a microphone for voice recognition, for input function. The present invention is not particularly limited in its functions by any input method.

The computer 2 is provided with data storage such as a hard disk, a semiconductor memory, a recording medium, or a memory card as a storage unit 310. The data may be a cloud service, a database, etc.

The standardized index creation module 211 of the computer 2 extracts at least the data for creating a standardized index from the user attribute data 101, status data 102, implementation data 103, immune status prediction data 104, and analysis result data 105 stored in the storage unit 310 (Step S51).

The standardized index creation module 211 of the computer 2 creates a standardized index from the extracted data (Step S52).

For example, the method of creating a standardized index may use rule-based or model-based machine learning to create a standardized index.

The standardized index provision module 212 of the computer 2 provides the created standardized index to a third-party company 5 (Step S53).

This is the standardized index creation process.

The computer (including CPU, an information processor, and various terminals) reads and executes a predetermined program to achieve the above-mentioned means and functions. For example, the program may be provided by one or more computers (SaaS: Software as a Service) through a network or a cloud service. The program may be provided in a form recorded in a computer-readable recording medium, for example. In this case, the computer reads a program from the recording medium, and forwards it to an internal or external storage, records it in the storage, and executes it. The program may be previously recorded in a storage (recording medium) and provided from the storage to the computer through a communication line.

The embodiments of the present invention are described above, but the present invention is not limited thereto. Moreover, the effects described in the embodiments of the present invention are only the most suitable ones produced from the present invention. The effects of the present invention are not limited to those described in the embodiments of the present invention.

### DESCRIPTION OF REFERENCE NUMERALS

1: System for providing immune status prediction, 2: Computer, 3: User terminal, 4: User, 5: Third party, 6: Network

What is claimed is:

1. A system for providing immune status prediction, comprising:
an acquisition unit that acquires at least one of the status data of sleep status, exercise status, lifestyle habit status, living status, working status, and dietary status related to an immune status;
a learning model creation unit that creates a learning model to generate immune status prediction data from the acquired status data;
a prediction unit that predicts immune status prediction data based on the learning model from newly acquired status data;
an analysis unit that generates analysis result data by analyzing the difference between the immune status prediction data and implementation data including a health checkup result and an immune test result;
a first output unit that outputs the predicted immune status prediction data;
a second output unit that outputs the generated analysis result data; and
a provision unit that provides at least one of the status data, the immune status prediction data, the implementation data, and the analysis result data to a user or a third party.

2. The system for providing immune status prediction according to claim 1, further comprising a learning model creation unit that creates a learning model that creates immune status prediction data from the implementation data and the previous status data.

3. The system for providing immune status prediction according to claim 2, wherein the provision unit provides at least one of the user attribute data, the status data, the immune status prediction data, the implementation data, and the analysis result data to a third-party company according to a user attribute preset by the third-party company.

4. The system for providing immune status prediction according to claim 2, further comprising:
a message generation unit that generates a message for joint development to the third-party company in response to a request received from the third-party company;
a message provision unit that provides the message to the third-party company; and
a message reception unit that receives the message from the third-party.

5. The system for providing immune status prediction according to claim 1 or 2, further comprising:
a standardized index creation unit that creates a standardized index from the user attribute data, the status data, the immune status prediction data, the implementation data, and the analysis result data; and
a standardized index provision unit that provides the standardized index to the third-party company.

6. A method for providing immune status prediction, the method being executed by a computer system, comprising the steps of:
acquiring at least one of the status data of sleep status, exercise status, lifestyle habit status, living status, working status, and dietary status related to an immune status;
creating a learning model to generate immune status prediction data from the acquired status data;
predicting immune status prediction data based on the learning model from newly acquired status data;
generating analysis result data by analyzing the difference between the immune status prediction data and implementation data including a health checkup result and an immune test result;
outputting the predicted immune status prediction data;
outputting the generated analysis result data; and
providing at least one of the status data, the immune status prediction data, the implementation data, and the analysis result data to a user or a third party.

7. A computer-readable program causing a computer system to execute steps of:
acquiring at least one of the status data of sleep status, exercise status, lifestyle habit status, living status, working status, and dietary status related to an immune status;
creating a learning model to generate immune status prediction data from the acquired status data;
predicting immune status prediction data based on the learning model from newly acquired status data;
generating analysis result data by analyzing the difference between the immune status prediction data and implementation data including a health checkup result and an immune test result;
outputting the predicted immune status prediction data;
outputting the generated analysis result data; and
providing at least one of the status data, the immune status prediction data, the implementation data, and the analysis result data to a user or a third party.
